(19) United States
(12) Patent Application Publication
(10) Pub. No.: US 2026/0158484 A1
(43) Pub. Date: Jun. 11, 2026

(54) METHOD FOR TREATING A CATALYST

(71) Applicant: Evonik Oxeno GmbH & Co. KG, Marl (DE)

(72) Inventors: Robert Franke, Marl (DE); Thomas Quandt, Marl (DE); Johannes Knossalla, Everswinkel (DE)

(73) Assignee: Evonik Oxeno GmbH & Co. KG, Marl (DE)

(21) Appl. No.: 19/104,061

(22) PCT Filed: Jun. 30, 2023
(86) PCT No.: PCT/EP2023/068057

§ 371 Date: Feb. 14, 2025

(30) Foreign Application Priority Data
Aug. 19, 2022 (EP) 22191216.5

(51) Int. Cl. B01J 38/48; B01J 21/12; B01J 23/78; B01J 23/94; B01J 35/61; B01J 37/02; B01J 37/10; B01J 38/02; C07C 2/24

(52) U.S. Cl. CPC B01J 38/485; B01J 21/12; B01J 23/78; B01J 23/94; B01J 35/615; B01J 37/0201; B01J 37/10; B01J 38/02; C07C 2/24; C07C 2523/755; C07C 2523/78

(57) ABSTRACT

A method for treating a catalyst may involve a hydrothermal treatment step in which the drying rate is lower than the drying rate in the subsequent drying step. The catalyst can be treated according to this method, before its first use, preferably in oligomerization, or after its use, preferably in oligomerization.

The present invention relates to a process for treatment of a catalyst, wherein the process comprises a hydrothermal treatment step where the drying rate is lower than in the subsequent drying. The catalyst may be treated with the process according to the invention prior to its first use, preferably in oligomerization, or after use, preferably in the oligomerization.

There are hardly any processes in industrial chemistry that do without catalyst. Some of the employed catalysts contain nickel as the active metal, for example in hydrogenation or oligomerization. Hydrogenation comprises (partially) saturating unsaturated carbon atoms by bonding hydrogen. Oligomerization comprises reacting unsaturated hydrocarbons with themselves in order thus to form the longer chain hydrocarbons known as oligomers (for example dimers, trimers or tetramers).

It is well-known that catalysts lose activity over time due to their use in chemical processes. This means that important parameters such as conversion or selectivity decrease over time. However, freshly produced catalysts may also have insufficient activity to be economically viable. The basic solution to this problem is a pretreatment of a fresh catalyst or a regeneration of a catalyst whose activity has fallen. Corresponding processes have been published in the literature numerous times.

In the case of industrial scale processes there is a perpetual object of improving the process. This includes the object of improving the employed catalysts and their activity to allow ever more economic operation of the processes on an industrial scale.

It is accordingly an object of the present invention to provide a process for treatment of a catalyst, preferably of an oligomerization catalyst, which allows the properties of the catalyst to be improved. Especially when used in oligomerization, higher selectivities and higher conversions in the oligomerization shall be achievable without any negative effect on the service life of the catalyst and the mechanical properties such as strength.

The problem addressed by the present invention was solved with the process for treatment of a catalyst according to claim 1. Preferred embodiments are specified in the dependent claims.

The process according to the invention is accordingly a process for treatment of a nickel-containing catalyst, wherein the process comprises at least the following steps:
a) thermal treatment of the catalyst at a temperature of 500° C. to 900° C. to remove carbon-containing deposits;
b) impregnation of the catalyst with an aqueous or ammoniacal solution comprising a nickel compound;
c) hydrothermal treatment of the catalyst, wherein the hydrothermal treatment is carried out in that the catalyst is heated from room temperature to a temperature in the range from 75° C. to 150° C., preferably 80° C. to 110° C. and optionally held at the temperature until a residual moisture content of not more than 30%, preferably of not more than 50% is obtained;
d) drying of the catalyst, wherein the drying rate in the drying in step cd) is higher than in the hydrothermal treatment in step bc) and wherein the drying is carried out to a residual moisture content of not more than 15%, preferably to a residual moisture content of not more than 10%; and
e) calcination of the catalyst.

The oligomerization catalyst has a composition of 15% to 50% by weight, preferably 15% to 40% by weight, of NiO, 10% to 30% by weight of Al₂O₃, 55% to 70% by weight of SiO₂ and 0.01% to 2.5% by weight, preferably 0.01% to 2% by weight, of an alkali metal oxide, preferably sodium oxide. The figures are based on a total composition of 100% by weight. In a particularly preferred embodiment of the present invention, the oligomerization catalyst is substantially free from titanium dioxide and/or zirconium dioxide, the oligomerization catalyst in particular comprising less than 0.5% by weight, preferably less than 0.1% by weight, particularly preferably less than 0.01% by weight, of titanium dioxide and/or zirconium dioxide in its total composition.

According to the invention the oligomerization catalyst may additionally have a specific surface area (calculated according to BET) of 150 to 400 m²/g, preferably 190 to 350 m²/g, particularly preferably of 220 to 330 m²/g. The BET surface area is measured by nitrogen physisorption according to DIN ISO 9277 (2014-01 version).

In a further preferred embodiment the oligomerization catalyst comprises mesopores and macropores, i.e. has a bimodal pore size distribution. The mesopores of the oligomerization catalyst according to the invention have an average pore diameter of 5 to 15 nm, preferably of 7 to 14 nm, particularly preferably of 9 to 13 nm. By contrast the macropores of the oligomerization catalyst according to the invention preferably have an average pore diameter of 1 to 100 μm, particularly preferably of 2 to 50 μm. The average pore volume of the oligomerization catalyst according to the invention, i.e. of both the mesopores and the macropores, may be 0.5 to 1.5 cm³/g, preferably 0.7 to 1.3 cm³/g. The average pore diameter and the average pore volume may be determined by mercury porosimetry according to DIN 66133 (1993-06 version).

The oligomerization catalyst according to the invention is preferably in the form of a granulate. Furthermore the oligomerization catalyst according to the invention may have an average particle diameter (d50) of 0.1 mm to 7 mm, preferably 0.5 to 6 mm, particularly preferably of 1 mm to 5 mm. The average particle diameter may be determined by imaging methods, and may be determined in particular by the methods cited in the standards ISO 13322-1 (2004 Dec. 1 version) and ISO 13322-2 (2006 Nov. 1 version 2006 Nov. 1) genannten Verfahren. A suitable device for analysing the particle diameter is for example the Camsizer 2006 (Retsch Technology).

In a further preferred embodiment the oligomerization catalyst has a bulk crush strength (BCS) of more than 0.5 MPa, preferably of more than 0.6 MPa and particularly preferably of more than 0.8 MPa. The BCS value is a measure of the mechanical strength of mineral granulates. The bulk crush strength (BCS) of a solid is to be understood as meaning a parameter defined as a pressure in MPa at which 0.5% by weight of fines fraction (i.e. particles screened off using a screen with a mesh size of 0.425 mm) are formed when the solid sample is subjected to pressure via a piston in a tube. For this purpose 20 ml of the solid are prescreened with a screen (mesh size: 0.425 mm), filled into a cylindrical sample tube (internal diameter: 27.6 mm, wall thickness: 5 mm, height: 50 mm) and 5 ml of steel spheres (diameter: 3.9 mm) are placed on the top surface of the solid. The solid is subsequently subjected to different (increasing) pressures for three minutes. The fines fractions formed by the subjection to pressure are then removed by screening, in each case weighed as a sum total and the percentage fraction thereof is determined. This process is performed until an amount of 0.5% by weight of fines fraction is reached.

An oligomerization catalyst may also be characterized by means of its maximum poured density. In a preferred embodiment the oligomerization catalyst according to the invention has a maximum poured density of 0.1 to 2 g/cm³, preferably 0.2 to 1.5 g/cm³, particularly preferably of 0.3 to 1.0 g/cm³. Determination of poured density may be carried out via a measuring cylinder. The measuring cylinder is filled with a certain volume of the solid to be investigated, for example via a suitable metering apparatus such as the DR100 apparatus (Retsch) and the measuring cylinder is weighed. The maximum poured density may be determined from the weight and the volume. It may be necessary to subtract the residual moisture from the sample weight.

The oligomerization catalyst according to the invention is produced by a process comprising the following general steps:
1) mixing of the amorphous silica-alumina support material, the Al-containing and Si-free binder and at least a portion of a nickel source, optionally also an alkali source, and granulation of the thus-produced mixture;
2) treatment (impregnation) of the granulate produced in step a) with at least a portion of a nickel source and/or an alkali source provided that the entirety of the nickel source and/or the alkali source has not already been mixed with the silica-alumina support material and the Al-containing and Si-free binder in step a); and
c) calcination of the granulate to produce the oligomerization catalyst.

Corresponding processes and precise conditions are known and disclosed for example in EP 3 549 669 A1, EP 3 546 065 A1 or in EP 3 542 898 A1.

After production of the catalyst, said catalyst may be employed in the oligomerization of olefins. However, the catalyst may also be subjected to the process according to the invention after its production but before its first use where, while some of the production steps are repeated, an additional hydrothermal step is performed. Direct production of the catalyst using a hydrothermal treatment step does not form part of the present invention.

The process according to the invention may also be used for regenerating a catalyst previously employed in the oligomerization. A reduction in conversion and/or selectivity may be encountered with increasing usage time of the oligomerization catalyst in the oligomerization, for example due to deposition of organic compounds. The catalyst according to the invention may be be regenerated, i.e. have an improved oligomerization activity compared to its previous state, with the process according to the invention after use in the oligomerization reaction.

The individual steps shall be more particularly elucidated hereinbelow:

### Step a)

After use in oligomerization reactions the oligomerization catalyst may exhibit deposits of organic substances. Removal of these deposits is often advisable. Removal of at least a portion of the organic compounds deposited in the catalyst is preferably accomplished in step a) by thermal treatment (oxidation) to form carbon oxides and water. Step a) may be performed continuously or discontinuously in a furnace, for example in a rotary kiln or a shaft furnace. To this end the oligomerization catalyst is supplied to the furnace and preferably maintained at a predetermined furnace temperature of 500° C. to 900° C., particularly preferably of 600° C. to 850° C. Step a) typically employs combustion air. The combustion air used is preferably supplied in countercurrent and in addition further air is optionally blown into the granulate (oligomerization catalyst) via suitable inlets to ensure faster oxidation.

### Step b)

Step b) comprises impregnation of the catalyst obtained from step a) with an aqueous or an ammoniacal solution. In the context of the present invention impregnation is to be understood as meaning contacting of the catalyst with the aqueous or ammoniacal solution, wherein impregnation may be carried out for example by spraying until permanent occurrence of a liquid film on the surface (incipient wetness). The impregnation introduces at least the moisture content desired for the subsequent hydrothermal treatment.

It is preferable according to the invention when the aqueous or ammoniacal solution comprises a nickel compound. This makes it possible to deposit additional amounts of nickel on the oligomerization catalyst. In principle any soluble nickel compound such as nickel nitrate (Ni(NO₃)₂), nickel acetate (Ni(ac)₂), nickel acetylacetonate (Ni(acac)₂), nickel sulfate (NiSO₄) or nickel carbonate (NiCO₃) may be used therefor to produce an aqueous or ammoniacal nickel solution.

The use of NiHAC solution, i.e. ammoniacal Ni(CO₃) solutions, has proven particularly advantageous. Such solutions may be employed with nickel contents of 0.5% to 14% by weight, in particular of 2% to 10% by weight, very particularly of 4% to 8% by weight.

### Step c)

The impregnation is followed in step c) by a hydrothermal treatment. The hydrothermal treatment is carried out in that the catalyst is heated from room temperature to a temperature in the range from 75° C. to 150° C., preferably 80° C. to 110° C. and optionally held at the temperature until a residual moisture content of not more than 30%, preferably of not more than 50% is obtained. The term “residual moisture content” relates to the state of the catalyst prior to the hydrothermal treatment, in which the catalyst has a residual moisture content of 100%. The catalyst is thus not dried at all, or only dried slowly, by the hydrothermal treatment.

The hydrothermal treatment is the core of the present process and must be clearly delimited from the subsequent drying in step d). In the hydrothermal treatment, the drying rate (loss in mass of evaporable components such as water or ammonia per unit time (e.g. min) is significantly lower than in the drying in step d). The hydrothermal treatment in step c) may be carried out at atmospheric pressure or at a pressure higher than atmospheric pressure, but not under vacuum. The hydrothermal treatment is preferably carried out in an apparatus which is suitable therefor, in which the drying rate can be limited. The hydrothermal treatment in step c) may for example also be carried out in a closed vessel, as a result of which the drying rate based on the volume of the vessel is 0. If the hydrothermal treatment is performed in a closed vessel the pressure increases due to the evaporating water and the evaporating ammonia. For safety reasons it may be necessary in this case for the closed vessel to have a pressure relief valve to prevent the pressure from becoming too high.

Step d)

The impregnated oligomerization catalyst is subsequently dried in a suitable drying apparatus, for example a belt dryer with an air stream or else a conical dryer, at temperatures between 80° C. and 250° C., preferably between 100° C. and 220° C., and atmospheric pressure or else under vacuum. The drying in step d) has a higher drying rate than the hydrothermal treatment in step c). In a preferred embodiment, the drying apparatus and the apparatus for the hydrothermal treatment are not identical. Instead, the oligomerization catalyst that is not completely dried can be removed from the apparatus in which step c is carried out and introduced into the drying apparatus.

### Step e)

Step e) is the final calcination. The calcination of the oligomerization catalyst may be performed continuously or discontinuously in a suitable furnace, for example a shaft furnace or rotary kiln. In the case of a continuous calcination it is further preferable when a gas is passed through the oligomerization catalyst (granulate) in countercurrent. The gas employed may be air, nitrogen or a mixture thereof. The gas stream may be 0.2 to 4 m³ of gas per kg of granulate per hour and the inlet temperature of the gas may be from 400° C. to 900° C., preferably 450° C. to 750° C. In addition to this heat introduced via the gas, energy may be introduced by active heating of the walls of the furnace.

The calcination temperature in the furnace may be 400° C. to 900° C., preferably 450° C. to 850° C. This temperature may be maintained over several hours, preferably 0.5 to 20 hours, particularly preferably 1 to 10 hours, before the granulate is cooled. Cooling is preferably carried out in an air stream.

The oligomerization catalyst according to the invention/the catalyst produced or regenerated with the process according to the invention may be used in particular for the oligomerization of C3- to C6-olefins, preferably C3- to C5-olefins, particularly preferably C4-olefins, or olefin-containing input mixtures based thereupon. The olefins or olefin-containing input mixtures are employed as a reactant stream

Once the process according to the invention has been performed the catalyst may be used for oligomerization of olefins. The present invention thus also provides a process for oligomerization of C3- to C6-olefins, wherein an olefin-containing input mixture containing the C3- to C6-olefins is passed over a catalyst in at least one reaction zone, wherein the catalyst has been subjected to a treatment according to the inventive process.

Employed olefins for the process according to the invention include C3- to C6-olefins, preferably C3- to C5-olefins, particularly preferably C4-olefins, or olefin-containing input mixtures based thereupon which may also contain proportions of analogous alkanes. Suitable olefins are inter alia α-olefins, n-olefins and cycloalkenes. The olefins used as reactants are preferably n-olefins. In a particularly preferred embodiment, the olefin is n-butene. According to the invention the term “olefin-containing input mixtures based thereupon” is to be understood as encompassing any type of mixtures containing the relevant C3- to C6-olefins to be oligomerized in an amount which makes it possible to perform the oligomerization. The olefin-containing input mixtures preferably contain practically no further unsaturated compounds and polyunsaturated compounds such as dienes or acetylene derivatives. It is preferable to employ olefin-containing input mixtures containing less than 5% by weight, in particular less than 2% by weight, of branched olefins based on the olefin proportion. Also preferably employed are olefin-containing input mixtures containing less than 2% by weight of branched olefins, in particular iso-olefins.

Propylene (C3) is produced on an industrial scale by cracking of naphtha and is a commodity chemical which is readily available. C5 olefins are present in light petroleum fractions from refineries or crackers. Technical mixtures which comprise linear C4 olefins are light petroleum fractions from refineries, C4 fractions from FC crackers or steam crackers, mixtures from Fischer-Tropsch syntheses, mixtures from the dehydrogenation of butanes, and mixtures formed by metathesis or from other industrial processes. Mixtures of linear butenes suitable for the process according to the invention are obtainable from the C4 fraction of a steam cracker for example. Butadiene is removed in a first step. This is accomplished either by extraction or extractive distillation of the butadiene or by selective hydrogenation thereof. In both cases a virtually butadiene-free C4-cut is obtained, namely raffinate I. In the second step, isobutene is removed from the C4-stream, for example by production of methyl tert-butyl ether (MTBE) by reaction with methanol. Other options include the reaction of the isobutene from the raffinate I with water to afford tert-butanol or the acid-catalysed oligomerization of isobutene to afford diisobutene. As desired, the now isobutene-free C4-cut, raffinate II, contains the linear butenes and any butanes. The 1-butene may optionally still be removed by distillation. Both fractions, the one comprising but-1-ene or the one comprising but-2-ene, may be used in the process according to the invention.

In a further preferred embodiment C4-olefin-containing material streams are supplied to the process as olefin-containing input mixtures. Suitable olefin-containing input mixtures include inter alia raffinate I (butadiene-free C4-cut from a steam cracker) and raffinate II (butadiene-free and isobutene-free C4-cut from a steam cracker).

A further option for producing a suitable olefin-containing input mixture is that of subjecting raffinate I, raffinate II or a similarly constituted hydrocarbon mixture to hydroisomerization in a reactive column. This may afford inter alia a mixture consisting of 2-butenes, small proportions of 1-butene and possibly n-butane and also isobutane and isobutene.

The oligomerization is generally carried out at a temperature in the range from 50° C. to 200° C., by preference 60° C. to 180° C., preferably in the range from 60° C. to 130° C., and at a pressure of 10 to 70 bar, preferably of 20 to 55 bar. If the oligomerization is to be carried out in the liquid phase, the parameters pressure and temperature must to this end be chosen such that the reactant stream (the employed olefins or olefin-containing input mixtures) is in the liquid phase. The weight-based space velocities (reactant mass per unit catalyst mass per unit time; weight hourly space velocity (WHSV)) of the olefin-containing input mixture are in the range between 1 g of reactant per g of catalyst per h (=1 h⁻¹) and 190 h⁻¹, preferably between 2 h⁻¹ and 35 h⁻¹, particularly preferably between 3 h⁻¹ and 25 h⁻¹. However, typical conditions are also known to those skilled in the art.

The oligomers produced by the process according to the invention are utilized inter alia for producing aldehydes, alcohols and carboxylic acids. Thus for example the dimerizate of linear butenes affords a nonanal mixture by hydroformylation. This provides either the corresponding carboxylic acids by oxidation or a C₉ alcohol mixture by hydrogenation. The Co acid mixture may be used for producing lubricants or siccatives. The C₉ alcohol mixture is a precursor for the production of plasticizers, in particular of diisononyl phthalates, diisononyl terephthalates, diisononyl cyclohexane-1,4-dicarboxylates or diisononyl cyclohexane-1,2-dicarboxylates.

The present invention is more particularly elucidated hereinbelow with reference to examples. Alternative embodiments of the present invention are obtainable analogously

### EXAMPLES

### Original Catalyst Material

The catalyst material derives from a production plant for oligomerization of butenes and has a typical composition of about 20% by weight NIO on Al₂O₃/SiO₂. The used catalyst material is initially regenerated such that the catalyst material is in a first step subjected to thermal treatment in a rotary furnace at temperatures between 550° C. and 650° C., in a second step subjected to post-impregnation with a 5% Ni solution, in a subsequent step dried at about 110° C. to 120° C. for more than 10 hours in a drying cabinet to a residual moisture content of approximately 15% and subsequently calcined at 650° C. in a tube furnace. No additional hydrothermal treatment was performed for the comparative catalyst 2. To produce the catalyst 1 according to the invention drying in the drying cabinet was preceded by a hydrothermal treatment. The material is subjected to hydrothermal treatment at 110° C. for 6 hours in a drying cabinet to a residual moisture content of approximately 30% in an only partially open container (closured with a venting screw cap with PTFE membrane), wherein the drying rate in the subsequent drying was higher. The two catalysts thus produced were employed for catalytic testing as follows.

About 350 g of the catalyst in each case were introduced into a metal tube having an internal diameter of 21 mm. Added in front of and behind the catalyst were glass beads having a diameter of 2 mm, which serve as a preheating and cooling phase. The oligomerization was performed using a feed stream at 30 bar and a loading of 2 g/h of butene per gram of catalyst, wherein the reaction temperature was varied between 80° C. and 100° C. The products were analysed by gas chromatography for the conversion of butenes and the linearity of the octenes. The compositions of the feed stream for the oligomerization are shown in Table 1 below.

TABLE 1
Composition of feed stream
Feed stream

| | |
|---|---|
| Isobutane | 8.0% |
| n-Butane | 15.3% |
| trans-2-Butene | 27.9% |
| 1-Butene | 32.7% |
| Isobutene | 0.9% |
| cis-2-Butene | 15.2% |

The conversions achieved for the feed stream as a function of temperature for catalyst 1 (inventive) and catalyst 2 (noninventive) and the ISO indices resulting therefrom are reported in table 2.

The linearity of an oligomerization product or of the dimers formed is described by the ISO index and represents a value for the average number of methyl branches in the dimer. For example (for butene as the reactant), n-octenes contribute 0, methylheptenes contribute 1 and dimethylhexenes contribute 2 to the ISO index of a C8 fraction. The lower the ISO index, the more linear the structure of the molecules in the respective fraction.

The ISO index is calculated by the following general formula:

$$\frac{( singly branched dimers ( wt \% ) + 2 \times doubly branched dimers ( wt \% ) )}{100}$$

Accordingly, a dimer mixture having an ISO index of 1.0 has an average of precisely one methyl branch per dimeric molecule. The proportion of the individual isomers is determinable by gas chromatography for example.

TABLE 2
Catalyst results

| | Conversion [%] | | Iso Index | |
|---|---|---|---|---|
| Temperature | Catalyst 1 | Catalyst 2 | Catalyst 1 | Catalyst 2 |
| 80° C. | 62 | 53 | 0.93 | 0.95 |
| 90° C. | 61 | 51 | 0.92 | 0.92 |
| 100° C. | 58 | 47 | 0.88 | 0.88 |

It is apparent that the inventive catalyst 1 in some cases achieves markedly higher conversions at comparable or lower ISO indices. This is surprising to the extent that a higher conversion is often accompanied by a lower linearity of the oligomers in the product mixture. The additional hydrothermal treatment thus ensures enhanced effectiveness of the catalyst, irrespective of temperature.

1-14. (canceled)

15. A process for treatment of a nickel-containing catalyst, the process comprising at least:
a) thermally treating the nickel-containing catalyst at a temperature of 500° C. to 900° C. to remove carbon-containing deposits;
b) impregnating the nickel-containing catalyst with an aqueous or ammoniacal solution comprising a nickel compound;
c) hydrothermally treating the nickel-containing catalyst, wherein the hydrothermal treatment is carried out in that the nickel-containing catalyst is heated from room temperature to a temperature in a range from 75° C. to 150° C. and optionally held at the temperature until a residual moisture content of not more than 30% is obtained, wherein the hydrothermal treatment is carried out at a pressure higher than atmospheric pressure;
d) drying of the nickel-containing catalyst, wherein a drying rate in the drying in d) is higher than in the hydrothermal treatment in c) and wherein the drying is carried out to a residual moisture content of not more than 15%; and
e) calcinating the catalyst.

16. The process according to claim 15, wherein the treatment process is carried out subsequently to a production of the nickel-containing catalyst or to a regeneration of the nickel-containing catalyst after employed in a heterogeneously catalyzed reaction.

17. The process according to claim 15, wherein the nickel compound is selected from the group consisting of nickel nitrate (Ni(NO₃)₂), nickel acetate (Ni(ac)₂), nickel acetylacetonate (Ni(acac)₂), nickel sulfate (NiSO₄), nickel citrate and nickel carbonate (NiCO₃).

18. The process according to claim 15, wherein the aqueous or ammoniacal solution employed is an ammoniacal Ni(CO₃) solution.

19. The process according to claim 15, wherein the thermal treatment in a) is carried out at a temperature of 600° C. to 850° C.

20. The process according to claim 15, wherein a duration of the hydrothermal treatment in c) is 1 to 24 hours.

21. The process according to claim 15, wherein the drying in d) is carried out at a temperature in a range from 80° C. to 250° C.

22. The process according to claim 15, wherein the drying in d) is performed such that an obtained dried material has a loss on drying (LOD) at 110° C. of not more than 5% by weight.

23. The process according to claim 15, wherein a drying rate in d) is twice as high as in c).

24. The process according to claim 15, wherein the calcination in e) is performed at a temperature between 400° C. and 900° C.

25. The process according to claim 15, wherein the calcination in e) is performed in an air stream, in a nitrogen stream or in a combination thereof.

26. The process according to claim 15, wherein the nickel-containing catalyst has a composition of 15% to 50% by weight of NiO, 10% to 30% by weight of Al₂O₃, 55% to 70% by weight of SiO₂ and 0.01% to 2.5% by weight of an alkali metal oxide.

27. The process according to claim 15, wherein the nickel-containing catalyst bas a specific BET surface area of 150 to 400 m²/g determined by nitrogen physisorption.

28. A process for oligomerization of C3- to C6-olefins, wherein an olefin-containing input mixture containing the C3- to C6-olefins is passed over a catalyst in at least one reaction zone, wherein the catalyst has been subjected to a treatment according to claim 15.
